(19) United States
(12) Patent Application Publication
(10) Pub. No.: US 2015/0184212 A1
(43) Pub. Date: Jul. 2, 2015

(54) Advanced Methods for Sugar Production from Lignocellulosic Biomass and Fermenting Sugars to Microbial Lipids

(71) Applicant: Washington State University, Pullman, WA (US)

(72) Inventors: Mahesh Bule, Pullman, WA (US); Allan Gao, Pullman, WA (US); Shulin Chen, Pullman, WA (US)

(21) Appl. No.: 14/410,769

(22) PCT Filed: Jun. 25, 2013
(86) PCT No.: PCT/US2013/047481

§ 371 Date: Dec. 23, 2014

Related U.S. Application Data
(60) Provisional application No. 61/666,163, filed on Jun. 29, 2012.

(51) Int. Cl. C12P 19/14; D21C 1/08; D21C 11/10; C12P 19/02

(52) U.S. Cl. CPC C12P 19/14; C12P 19/02; D21C 1/08; D21C 11/10; C12P 2201/00

(57) ABSTRACT

Methods for facilitating sugar release from lignocellulosic biomass and for utilizing the sugars for microbial lipid (e.g. biofuel) production are provided. The methods involve pretreating lignocellulosic biomass using various oxidizing agents (ozone, peroxone, etc.) at a temperature not higher than 50° C. and pressure no higher than 1.5 atm to render the biomass more accessible to enzymatic hydrolytic degradation into sugars and utilizing soluble sugars for fermenting oleaginous microorganism to produce microbial lipids.

### FIELD OF THE INVENTION

This invention relates to the development of an effective method for obtaining sugars from lignocellulosic biomass and utilizing said sugars for fermentation to biofuel and biochemical such as microbial lipid. In particular, the invention provides methods for pretreating lignocellulosic biomass, hydrolyzing pretreated biomass using different enzyme cocktails at a high solid loading, utilizing the enzymatic hydrolysate containing released sugars for production of microbial lipids using oleaginous yeast fermentation, and production of microbial lipids with simultaneous saccharification (by lignocellulose hydrolyzing enzymes) and fermentation (by lipid producing oleaginous yeast).

### BACKGROUND OF THE INVENTION

Lignocellulosic biomass such as agricultural and forestry residues are attractive feedstocks for biofuel and biochemical production because of the availability of a large supply at low cost (Cardona & Sánchez, 2007) and the added benefit of environmental sustainability (Demirba [symbol] , 2003). Lignocellulose is composed of an intricate network of cellulose, hemicellulose and lignin, and the composition varies according to type, species and even sourced location (Carere et al., 2008; Chandra et al., 2007). Due to the recalcitrant nature of these complex polymers an intensive thermochemical treatment (i.e. a “pretreatment”) is required (Himmel et al., 2007) to produce a substrate which can be easily hydrolyzed by commercial cellulolytic enzymes, or by enzyme producing microorganisms, to release sugar for fermentation.

Different sources of lignocellulosic biomass inherit a differential degree of recalcitrance to cellulolytic enzymes. Factors such as lignin content, cellulose crystallinity and packing structure, and hemicellulose branching determines the overall ability of biomass to be saccharified. Lignin removal (Jeoh et al., 2007) and reduction of crystallinity (Mosier et al., 2005) of cellulose enhances biomass digestibility, and can be achieved with pretreatment. However, the pretreatment step is one of the most significant cost factors in the conversion process of lignocellulosic biomass to biofuels or biochemicals. Acceptable pretreatment technologies should accomplish high sugar recovery with low enzyme consumption, low cost, and minimal carbohydrate degradation. In addition, the process should not produce high concentrations of toxicants that may inhibit downstream processes including saccharification and fermentation (Lynd et al., 1999; Tao et al., 2011). Various pretreatment methods including dilute acid, steam explosion, hot water, ammonia fiber explosion, alkaline hydrolysis, oxidative delignification, organosolv, biological pretreatment and ozonation have been investigated and used to decrease the recalcitrance of biomass through modifying physico-chemical factors.

Extensive prior reports suggest the art of pretreating lignocellulosic biomass with the use of ozone. The following patents and literature references describe this process: U.S. Pat. No. 20100159521A1 (Cirakovic & Diner, 2010b); U.S. Pat. No. 20100159522A1 (Cirakovic & Diner, 2010a); E. P. Pat No. 0,045,500 (Ishibashi et al., 1985); (Garcia-Cubero et al., 2009; García-Cubero et al., 2012). All of these patents and publications are incorporated herein in their totalities.

The utilization of anhydrous liquid ammonia or ammonium hydroxide solution for pretreatment of lignocellulosic biomass is well practiced. The following patents and literature references describe this process: U.S. Pat. No. 20120325202 (Dale, 2012); (Kim & Lee, 2005,2007; Kim et al., 2008; Yoo et al., 2013). All of these patent and publications are incorporated herein in their totalities.

The above mentioned pretreatment methods suffer from shortcomings, including separate hexose and pentose streams (e.g. concentrated and dilute acid), degradation of sugars (e.g. to acid or aldehyde which results in poor carbohydrate recovery and inhibition to subsequent downstream processes such as saccharification and fermentation), long residence times (e.g. biological pretreatment), incomplete destruction of the lignin-carbohydrate matrix (e.g. steam pretreatment), and disposal of waste product through the neutralization of acid or base. Combinational pretreatment strategies are generally more effective in enhancing the biomass digestibility, and often employed in designing leading pretreatment technology. A method using a combination of alkaline treatment with a low concentration of anhydrous ammonia at higher temperatures (75-150° C.) followed by ozone and vice-versa has been reported to improve enzymatic saccharification (Cirakovic & Diner, 2010b). However, the exact function of either step was not clearly stated in this prior art. A pretreatment method for cotton straw with ammonium hydroxide at room temperature for 60 days followed by ozone treatment resulted in a 50% reduction of lignin (Ben-Ghedalia et al., 1980). The reaction rate of advanced oxidation processes performed using .OH (ozone/H₂O₂, peroxone) radicals are much higher than ozone alone because they react with organic as well as inorganic compounds speedily and overcomes the effect of diffusion-controlled limit (Nothe et al., 2009).

After pretreatment of biomass, it is generally processed to produce fermentable sugars either by separate or simultaneous saccharification using cellulase enzymes. For industrial biofuel processes high sugar concentrations after saccharification are preferred to develop continuous or fed batch fermentation process. Alternative to separate hydrolysis and fermentation (SHF), simultaneous saccharification and fermentation (SSF) processes are extensively used in ethanol fermentation (Elliston et al., 2013; Lim et al., 2013; Lin et al., 2013; Suriyachai et al., 2013) but have not been studied extensively for microbial lipid production by oleaginous microorganisms. Oleaginous microorganisms such as yeast and fungi have the ability to utilize C5 and C6 sugars derived from lignocellulosic biomass (Gao et al., 2013; Huang et al., 2009; Tsigie et al., 2011; Yu et al., 2011; Zeng et al., 2013), making it efficient and necessary to develop a pretreatment and saccharification process which is capable of producing a single sugar stream (containing C5 and C6 sugars) and test the applicability of these lignocellulose derived sugars for production of biofuel and bioproducts. Oleaginous microorganisms have capability to accumulate lipids up to 70% of the total dry cell weight (Chen et al., 2009). However, the requirement of heterotrophic culture is that an organic carbon source is primary need. Numerous reports suggest use of oleaginous yeasts for lipid accumulation on different substrates, such as glycerol (Duarte et al., 2013; Kitcha & Cheirsilp, 2013; Meesters et al., 1996; Papanikolaou & Aggelis, 2002), sewage sludge (Angerbauer et al., 2008; Huang et al., 2013a; Peng et al., 2013), whey permeate (Akhtar et al., 1998; Ykema et al., 1988), sugar cane molasses (Alvarez et al., 1992; Gonzalez-Garcia et al., 2013; Schneider et al., 2013) and rice straw hydrolysate (Huang et al., 2009). The utilization of lignocellulosic biomass is critical to avoid “fuel versus food” issue. The fatty acid profile of microbial lipid is analogous to that of conventional vegetable oils, oleaginous yeast has been suggested as a favorable microorganism for a sustainable biodiesel industry (Zhao et al., 2010).

### SUMMARY OF THE INVENTION

An exemplary embodiment of the invention provides a process by which lignocellulosic biomass can be pretreated; saccharified and soluble sugars/biomass suitable for fermentation can be obtained for biofuel and bioproduct production. This process is purposely designed as two steps. The first step targets mainly at breaking the lignin barrier at the surface of the cellulosic materials so that the hydrophobicity will be greatly decreased. The second step allows the removal of lignin from the secondary plant cell wall and swelling the fiber structure to facilitate the access of cellulase enzyme to hydrolyze the cellulose into sugar. Further, in some of the epitome process to improve the lignin modification or destruction step of the pretreatment process by the means of utilizing highly reactive reagents (such as hydrogen peroxide including but not limited to) in first step and improve efficiency of second step. Further, another embodiment describes the process to develop an enzymatic hydrolysis process that can produce soluble sugars using a high solid loading of pretreated lignocellulosic biomass. Further, certain embodiment provides process for utilizing pretreated biomass directly in the simultaneous saccharification and fermentation process to produce microbial lipids and/or utilizing it for development of separate enzymatic hydrolysis and fermentation process for microbial lipid production using oleaginous microorganisms (any species of yeast or fungi or bacteria or algae). These and other embodiments will become progressively clear by reference to the following delineations and drawings.

The methods include using lignocellulosic material which has been mechanically ground or milled and which contains a variable amount of moisture. The lignocellulosic material is then treated selectively by oxidizing reagents to modify the structure of lignin on the surface of the material, followed by lignocellulosic material swelling and de-crystallization of cellulose using aqueous ammoina. Following this treatment, the pretreated biomass is primarily structural carbohydrate, and is facile to enzymatically hydrolyze for sugar production. After pretreatment, the high carbohydrate content biomass is hydrolyzed with a cellulolytic enzyme cocktail to produce a single stream of soluble fermentable sugars including both C5 (xylose, mannose and arabinose) and C6 (glucose and galactose). This stream of sugars contains minimal amounts of pretreatment derived inhibitors such as organic acids, furfural, hydroxyl methyl furfural, etc. The obtained soluble fermentable sugars may be further processed and utilized for biochemical or bioproduct production.

In an embodiment, a process for pretreating lignocellulosic biomass comprises:

(a) providing lignocellulosic material containing cellulose, hemicellulose and lignin;

(b) grinding the lignocellulosic biomass, e.g. to about 20-60 mesh size;

(c) adjusting the moisture content of the biomass to 0% to 90% (e.g. with water);

(d) treating biomass with a gas containing ozone, wherein concentration of ozone is between 1 to 20% (w/w), in a stainless steel vessel with semi-continuous mode at a temperature of about 0° C. to 25° C. and for reaction time of about 1 to 20 minutes, whereby lignin present in the lignocellulosic biomass is modified to some extent;

(e) further treating the biomass by soaking it in an aqueous ammonia solution, where ammonia is present at a concentration of not more than about 30% (w/v) and the biomass solid loading into the ammonia solution is between about 1% to about 20% (w/v), for a reaction time of from about 1 to about 24 hours at 15° C. to 50° C. After the reaction, the aqueous fraction containing lignin and aqueous ammonia liquor is separated e.g. by filtration or centrifugation, and residual solids are washed and/or neutralized e.g. with the addition of acids such as sulphuric (H₂SO₄) or hydrochloric (HCl). The pretreatment process expands the cellulose microfibril structure and decreases the crystallinity of cellulose, resulting in a cellulosic biomass product that can be easily hydrolyzed into monomeric sugars.

In another embodiment, a process for pretreating lignocellulosic biomass comprises:

(a) providing lignocellulosic material containing cellulose, hemicellulose and lignin;

(b) grinding the lignocellulosic biomass e.g. to about 20-60 mesh size;

(c) forming or providing a hydrogen peroxide (H₂O₂) solution with a concentration of from about 3 to about 30% (w/v) hydrogen peroxide in water

(d) adjusting the biomass moisture content to from about 0% to about 90% with the hydrogen peroxide solution;

(e) treating moisture adjusted biomass with a gas containing ozone, wherein the concentration of ozone is between about 1 to about 20% (w/w), in a stainless steel vessel with semi-continuous mode at a temperature of from about 0° C. to about 25° C. for reaction time of from about 1 to about 20 minutes, whereby lignin present in the lignocellulosic biomass is modified to some extent;

(f) further treating the biomass by soaking it in an aqueous ammonia solution, where ammonia is present at a concentration not more than about 30% (w/v) and biomass solid loading into the ammonia solution is between about 1% to about 20% (w/v) for a reaction time of about 1 to about 24 hours at about 15° C. to about 50° C. After the reaction, the aqueous fraction containing lignin and aqueous ammonia liquor is separated e.g. by filtration or centrifugation and residual solids are washed or neutralized e.g. by the addition of acid such as sulphuric (H₂SO₄) and/or hydrochloric (HCl). The pretreatment process expands the cellulose microfibril structure and decreases the crystallinity of cellulose, resulting in a cellulosic biomass product that can be easily hydrolyzed into monomeric sugars.

In some embodiments, the methods for lignocellulosic biomass pretreatment includes:

After following steps (a) and (b) above, adjusting biomass moisture to from about 0% to about 90% (w/v) with water or with a solution of hydrogen peroxide in water (concentration about 3 to about 30%, w/v);

treating the moisture adjusted biomass with a gas containing ozone, wherein concentration of ozone is between from about 1 to about 20% (w/w), in a series of stainless steel vessels operated in semi-continuous mode at an ambient temperature between from about 0° C. to about 25° C. and for a reaction time of from about 1 to about 20 minutes, whereby lignin present in the lignocellulosic biomass is modified to some extent;

and further following step (f) for soaking aqueous ammonia as described above to produce saccharifiable biomass.

In another embodiment, a process for producing pretreated lignocellulose biomass comprises:

After following steps (a) and (b) above: adjusting the moisture to about 0% to 90% (w/v) with or without hydrogen peroxide (concentration 3-30%, w/v) addition to the water;

treating moisture adjusted biomass with a gas containing ozone, wherein concentration of ozone is between about 1 to about 20% (w/w), and biomass is treated either in a series of stainless steel vessels or in a single vessel operated in semi-continuous mode at an ambient temperature of between about 0° C. to about 25° C. and for a reaction time of about 2 to about 20 minutes, whereby lignin present in the lignocellulosic biomass is modified to some extent and the unreacted ozone or other gases generated during the reaction are recycled to produce a pure stream of air and oxygen for reuse;

and further following the previous step with the aqueous ammonia soaking process as described above to produce saccharifiable biomass.

In another embodiment, a process for recovering lignin and ammonia from spent liquor of ammonia after step (e) comprises:

collecting the solution of lignin-aqueous ammonia liquor (temperature about 30° C. to about 50° C.) into a reservoir;

evaporating ammonia from the lignin-aqueous ammonia liquor at a temperature between about 50° C. to about 80° C.;

recovering the ammonia in the form of ammonium hydroxide to be utilized again for soaking aqueous ammonia pretreatment;

recover lignin from the remaining liquor, which no longer contains ammonia, by evaporation, acid precipitation, or membrane filtration;

purifying recovered lignin with a process that includes (i) acetic acid solubilization, (ii) precipitation in excess water, (iii) centrifugation, (iv) solubilization in methylene chloride, (v) precipitation in diethyl ether, (vi) and finally drying to obtain pure lignin.

In another embodiment, the methods of the invention further comprise hydrolyzing pretreated biomass with an enzyme cocktail to produce soluble fermentable sugars. The hydrolysis process comprises forming a solution of biomass in buffer at a concentration of about 5% to about 20% (w/v), and enzymatically hydrolyzing the pretreated biomass (e.g. with a cocktail of microbes) to produce a concentrated stream of sugars.

In another embodiment, a process for utilizing soluble sugars for fermentation is disclosed. The soluble sugars may be obtained as described herein. The hydrolysate produced after the enzymatic reaction can be utilized for production of microbial lipids, ethanol, n-butanol, iso-butanol, or other biochemicals and bioproducts.

In another embodiment, a process for utilizing pretreated biomass directly for simultaneous saccharification and fermentation is disclosed. The pretreated biomass can be used in a simultaneous saccharification and fermentation process for biofuels and bioproducts at temperatures between about 25° C. and about 40° C.

Other features and advantages of the present invention will be set forth in the description of invention that follows, and in part will be apparent from the description or may be learned by practice of the invention. The invention will be realized and attained by the compositions and methods particularly pointed out in the written description and claims hereof.

### BRIEF DESCRIPTION OF THE DRAWINGS

For detailed understanding and clarity of the invention, reference should be made to the following comprehensive description and accompanying drawings wherein:

FIG. 1 is a process flow diagram for two-step ozone and soaking aqueous ammonia pretreatment process 100.

FIG. 2 is a process flow diagram for two-step ozone and soaking aqueous ammonia pretreatment process where the ozonation process 102 is performed in a series of reactors.

FIG. 3 is a process flow diagram for a two-step ozone and soaking aqueous ammonia pretreatment process where the gas stream is cleaned by scraping unwanted gases and remaining ozone with a scraper to reduce the cost of air/gas in the process 103.

FIG. 4 is a process flow diagram for recovery of ammonia and lignin in process 104.

FIG. 5 is a process flow diagram for a separate hydrolysis process 200 and fermentation for microbial lignin production process 300 using oleaginous microorganisms.

FIG. 6 is a process flow diagram for simultaneous saccharification and fermentation process 400 for microbial lipid production using oleaginous microorganisms.

FIG. 7 represents microbial lipid production by different strains of oleaginous yeast

FIG. 8 shows mass balance data for simultaneous saccharification and fermentation of wheat straw particles in association with ozone followed by soaking aqueous ammonia (OSAA) pretreatment process.

### DETAILED DESCRIPTION

Lignocellulosic biomass materials comprised primarily of structural carbohydrates or polysaccharides (cellulose and hemicellulose) has the potential to supply sugars as a renewable substrate for biofuel (bioethanol, bio-butanol, microbial lipid, biomethane and the like) and biochemical by fermentation or non-biological transformation route at a low-cost. However, lignocellulose-based substrates are a highly recalcitrant material that requires an intensive pretreatment process before the polysaccharides can be accessed. The present invention provides a systematic approach for pretreating lignocellulosic biomass in two steps. Initially the hydrophobic lignin molecule is targeted with highly reactive oxidizing agents (such as ozone, peroxone and the like) and then the partially treated biomass is further pretreated with a relatively low temperature, low pressure process by soaking the biomass in an aqueous ammonia solution. This disclosure also includes methods to minimize the amount of pretreatment reagents (ozone, hydrogen peroxide, ammonia) utilized and/or for recycling the reagents. The methods are advantageous in part due to the use of low temperature and pressure conditions.

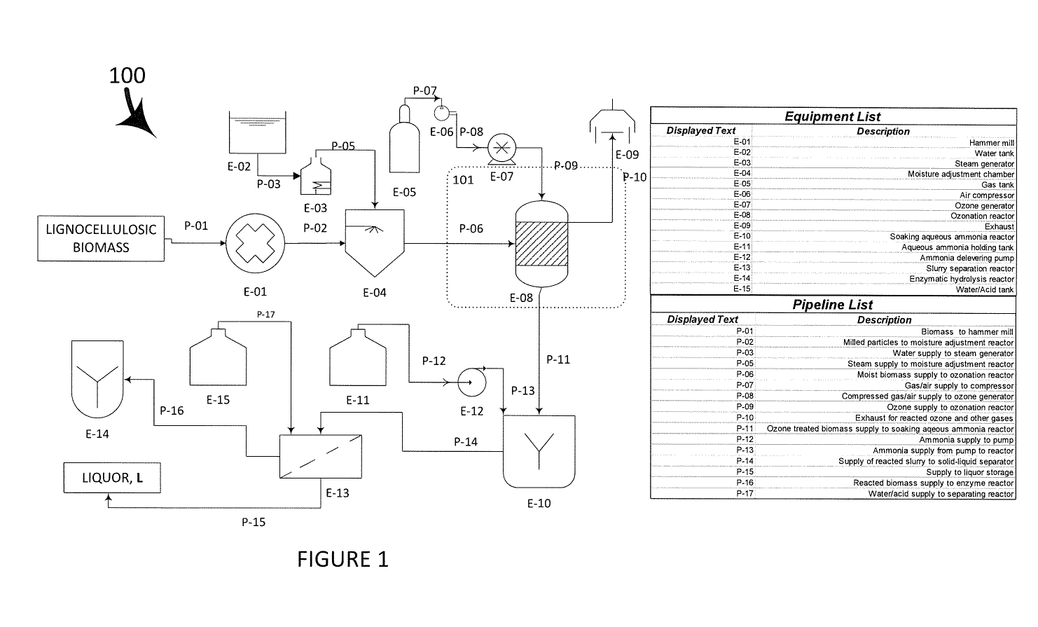

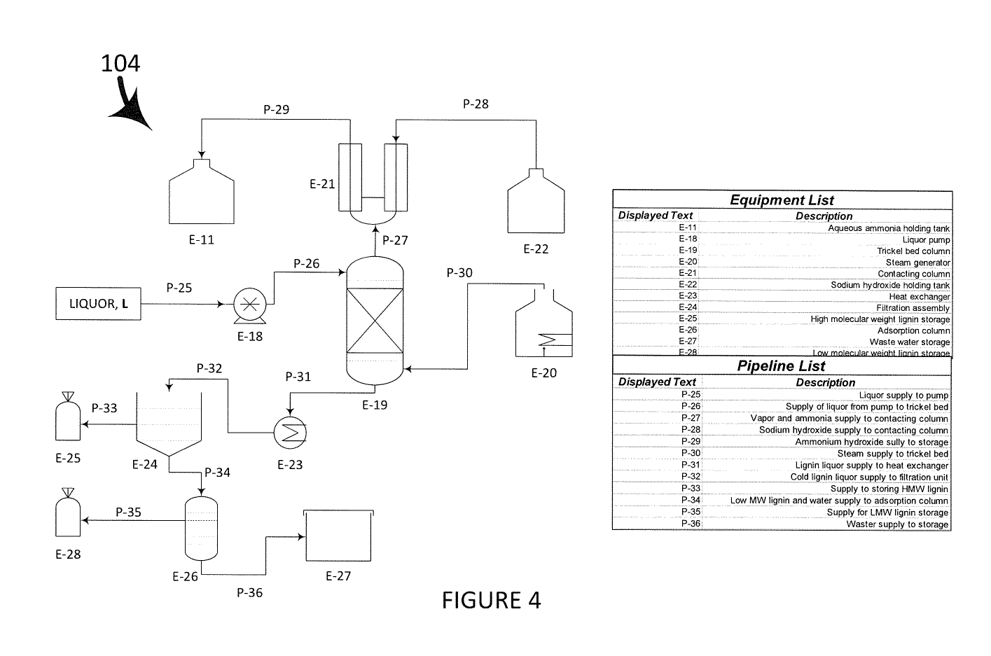

The basic pretreatment process 100 to the present invention is represented by the flow diagram in FIG. 1. Lignocellulosic “biomass” is milled in a hammer mill E-01 and passed to moisture adjustment reactor E-04 through supply P-02. Moisture adjustment is performed using steam generator reactor E-03. The resulting moisture adjusted biomass is then provided as feed to pretreatment process 101. Here, in some of the embodiments, hydrogen peroxide is added with water in tank E-02. A gas stream (from storage chamber E-05) of pure oxygen, an oxygen-air mixture, or purified air is fed into compression system E-06 through air supply P-07. Compressed gas stream (P-08) in supplied to ozone generator E-07. The gas enriched with ozone is then supplied through pipe P-09 to pretreatment reactor E-08 in which moist biomass is already charged. After the pretreatment reaction, the excess gas mixture leaves to exhaust E-09 through connector P-10 and the treated biomass is transferred to a soaking aqueous ammonia pretreatment in reaction vessel E-10 through pipe P-11. The required aqueous ammonia solution for pretreatment is supplied either as fresh from reservoir E-11 with the use of piping and pumping system (P-12, P-13 and E-12) or from recycle process 104 (shown in FIG. 4) through the same system. The slurry of treated biomass is then injected into the separating vessel E-13 through pipe P-14, and the separated biomass is either washed with fresh water from tank E-15 or residual ammonia is then neutralized by acid supplied from reservoir E-15. The separated liquor from vessel E-13 is further processed by process 104 (shown in FIG. 4). More reactive biomass from reactor E-13 is then used as feed to process 200 to obtain soluble fermentable sugars which could be used for producing biofuels or bioproducts in the process 300 or biomass could directly be used for the process 400.

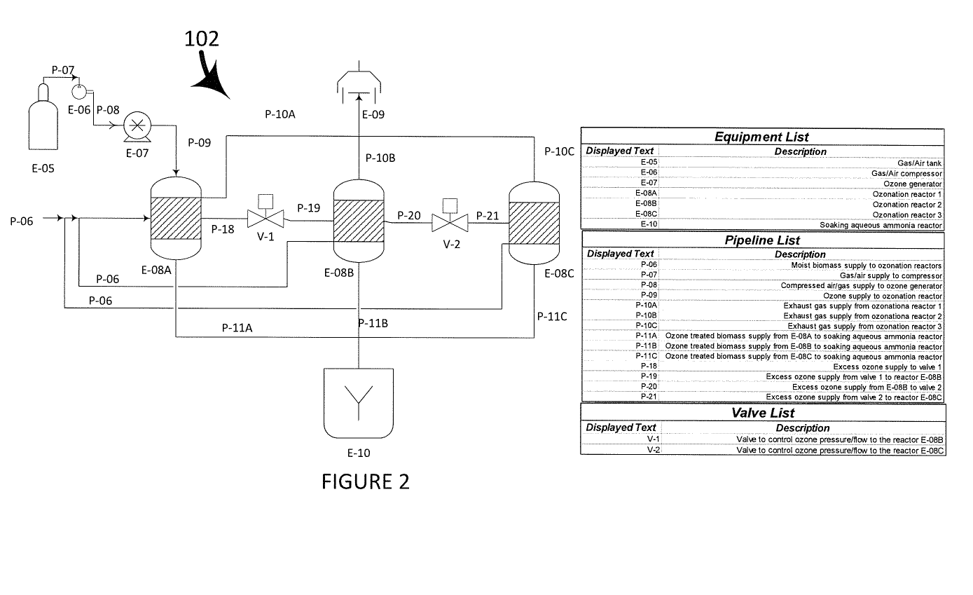

FIG. 2 Illustrates modifications to the process 101 in order to reduce its cost by utilizing ozone more efficiently in the series of reactors (E-08A and/or E-08B and/or E-08C). The specific modification in process 102 including the gas enriched with ozone is being passed through vessel E-08A and its exhaust is connected/not connected to vessel E-08B through piping and valve system (P-18, P-19 and V-1) as a feed, similarly connected/not connected to vessel E-08C through piping and valve system (P-20, P-21 and V-2) as a feed.

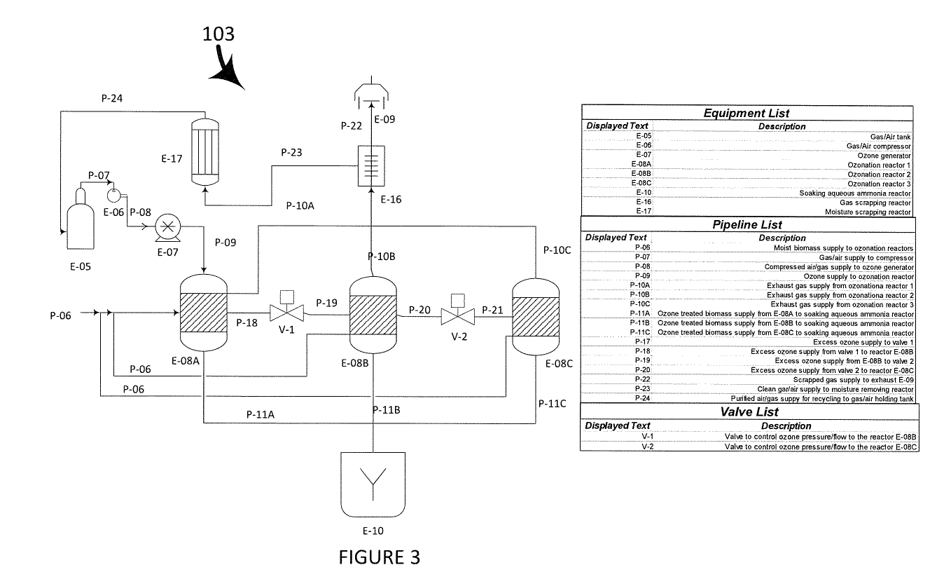

FIG. 3 Exemplifies modifications to processes 101 and 102 to reduce feed gas cost by employing gas clean-up system E-16. Gas clean-up systems may contain a scrubber and/or thermal destruct and/or catalytic destruct which are previously described in earlier reference as follows: U.S. Pat. No. 6,315,861B1 (Joseph et al., 2001) followed by gas conditioning unit E-17 which contains a cooling unit and/or desiccant unit. Gas clean-up systems are commercially available and can be easily understood by persons skilled in the art.

FIG. 4 Shows a further process 104 which utilizes methods for handling liquor, L obtained during process 100. Initially, liquor is being pumped and supplied to the trickle bed vessel E-19 available in market commercially, using pump E-18 and pipes P-25 and P-26. Then steam is passed through vessel E-19 from the bottom to have a counter current effect for stripping most of the ammonia from the liquor. One skilled in the art will know how to operate stripping system for ammonia. The vapor containing ammonia is then being contacted with sodium hydroxide (supplied from vessel E-22) to be converted in to ammonium hydroxide again in the contacting vessel E-21. The recycled ammonia is then being stored in vessel E-11 for further pretreatment cycle of soaking aqueous ammonia. The down-comer stream containing water, lignin, silica, extractives etc. is then being passed through the heat exchanger E-23 to recover heat, and the cooled stream is being supplied to the separating vessel E-24. After either sieving or membrane separation high molecular weight lignin is being recovered and stored into vessel E-25. The stream generated from separating vessel E-24 is supplied to the adsorption column to adsorb low molecular weight lignin and is stored in vessel E-28. The waste stream from column is further received into storage tank E-27.

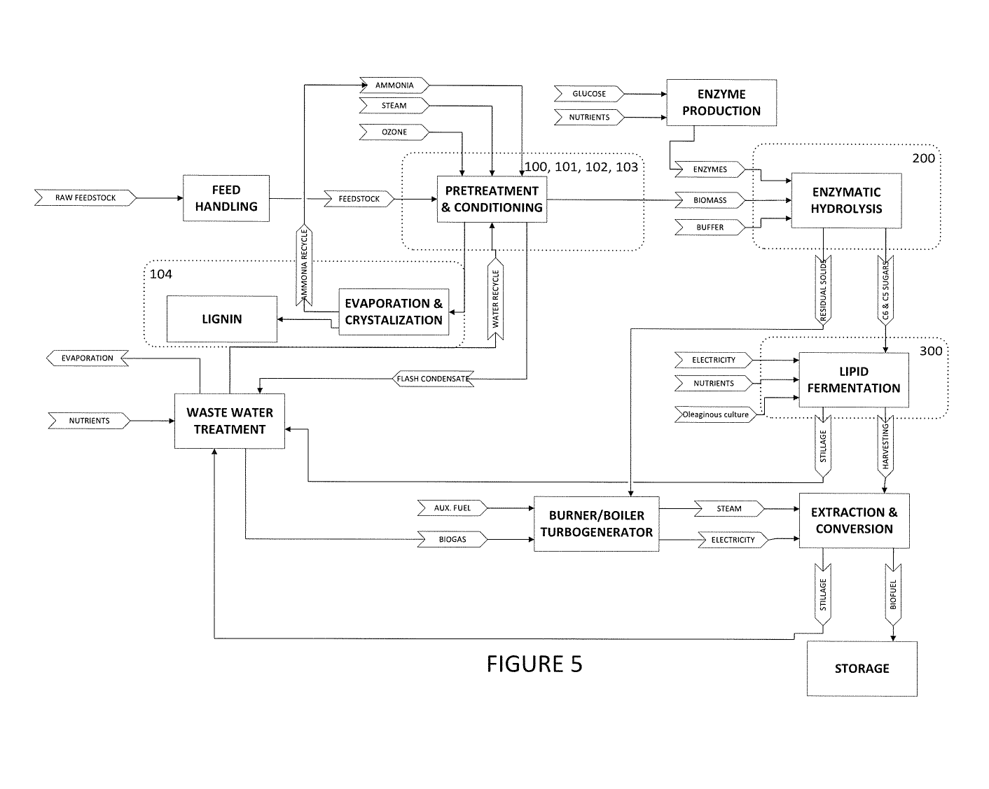

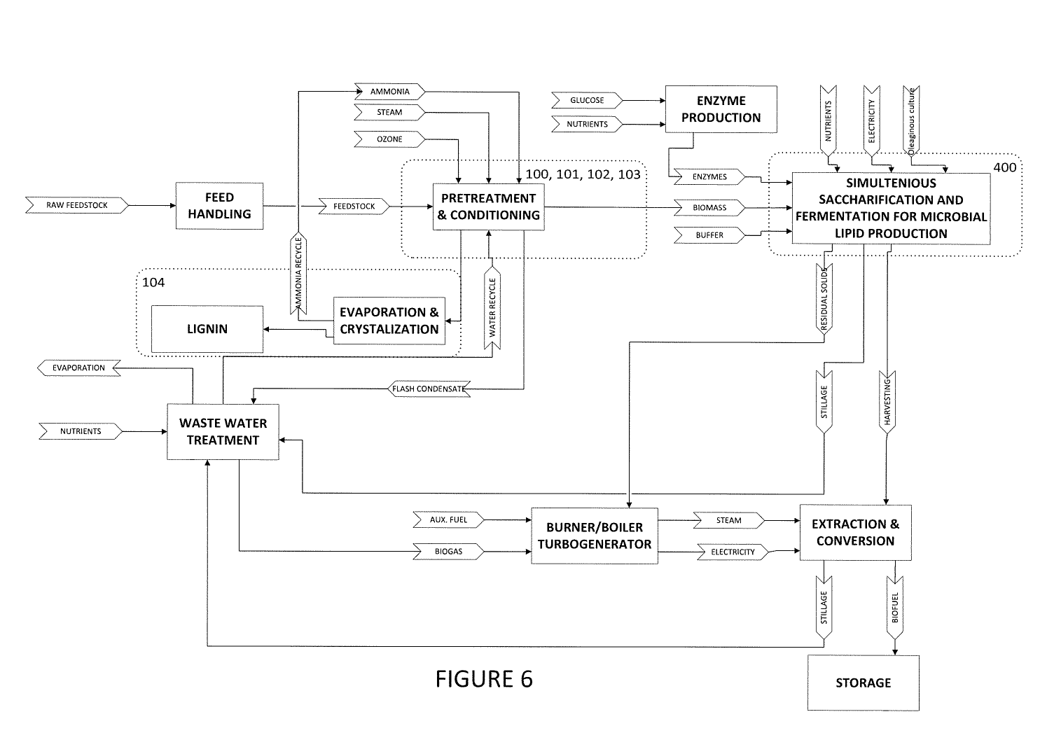

FIGS. 5 and 6 show further processing of treated lignocellulosic biomass in enzymatic hydrolysis process 200, lipid fermentation process 300 and simultaneous saccharification and fermentation process 400. Further processing includes the pretreated lignocellulosic material obtained from process 101 and/or 102 and/or 103 is being treated with one or more hydrolyzing enzymes in the process 200. Potentially, the treatment is performed in an aqueous medium containing the lignocellulosic biomass, the saccharifying enzymes, a buffer solution of either citrate or acetate, and water. For solid loading during saccharification, suitable glucan can be loaded in aqueous hydrolysis medium for example ranging from about 0.1% (w/v) to 20% (w/v). One skilled in the art will know about the term glucan content of the lignocellulosic biomass. The stream of soluble sugars (e.g. glucose, xylose, arabinose, mannose etc.) can be obtained at the end of process 200. This stream is then being further utilized for fermentation process 300, here in this invention oleaginous yeast are utilized as exemplary, and are not limited to them, while a range of microorganisms utilizing sugars can be used for various processes such as bioethanol, biobutanol, organic acids, antibiotics etc. In another process 400, the pretreated biomass is being directly used in a simultaneous saccharification and fermentation process.

### DEFINITIONS

“Lignocellulosic” refers to a material primarily comprising lignin, cellulose, and hemicellulose. “Lignocellulosic biomass” refers to plant material containing lignin, cellulose, and hemicellulose, including but not limited to wheat straw, barley straw, rice straw, corn stover, sugarcane, bagasse, grasses (e.g. switchgrass), hemp, corn, sorghum, sugarcane, bamboo, trees and wood (e.g. eucalyptus, oil palm, poplar, willow, Miscanthus giganteus, etc.), waste paper, newspapers, nut shells, manure, municipal waste solids, and the like. Lignocellulosic biomass can be broadly classified into virgin biomass, waste biomass and energy crops. Virgin biomass includes all naturally occurring terrestrial plants such as trees, bushes and grass. Waste biomass is produced as a low value byproduct of various industrial sectors such as agricultural (corn stover, sugarcane bagasse, straw etc), forestry (saw mill and paper mill discards). Energy crops are crops with high yield of lignocellulosic biomass produced to serve as a raw material for production of second generation biofuel; examples include switch grass (Panicum virgatum) and Elephant grass.

“Carbohydrate” refers to long chain sugar molecules or polysaccharides found in lignocellulosic biomass, including cellulose and hemicellulose.

“Sugars” refers to sugars obtained from hydrolysis of lignocellulosic biomass. These sugars include glucose, xylose, mannose, arabinose, and galactose.

“Pretreatment” refers to a process used to treat lignocellulosic biomass prior to enzymatic hydrolysis. The methods disclosed herein involve pretreating biomass in order to render it more amenable to hydrolysis, e.g. enzymatic hydrolysis. The agents used in the pretreatment include one or more of: one or more highly reactive oxidizing agents and one or more alkalinizing agents. The pretreatment methods steps are generally carried out under reduced (low) temperature and and/or under reduced (low) pressure. In some aspects, only reduced temperature is employed. In other aspects, only reduced pressure is employed. In yet other embodiments, both low temperature and low pressure are employed. By “low temperature” we mean temperatures that are at most about 30° C., and which may be as low as 4° C. Thus, temperatures in the range of from about 4° C. to about 30° C. are used. By “carried out under low pressure” we mean that the pretreatment reactions are carried out under conditions of pressure up to 1.5 atm. Advantages of conducting these reactions under conditions of reduced temperature and/or pressure include, for example: low cost of maintenance, operation and capital cost.

Once suitably sized as described in some of the embodiments, the biomass is introduced into a vessel/container for carrying out the reaction. The container must be capable of maintaining the desired temperatures and pressures described herein, and may further include mixing or stirring means, various gauges for monitoring temperature and pressure, various inlet and outlet valves for introducing and for removing (e.g. siphoning off) reactants, etc. The vessel should also be fabricated of material that is not susceptible to easy corrosion when exposed to the agents employed herein, e.g. stainless steel.

The biomass is then contacted with (exposed to) at least one oxidizing agent. Exemplary oxidizing agents that may be used in the practice of the invention include but are not limited to: ozone; peroxone (ozone plus hydrogen peroxide); hydrogen peroxide alone, enzymes such as laccase, peroxidase etc. and combinations thereof. By “combinations” we mean that the biomass may be exposed to two or more oxidizing agents at the same time (e.g. in a single mixture that contains biomass and the two or more agents) or sequentially, i.e. the biomass may be exposed first to one oxidizing agent and then to another different oxidizing agent. In some aspects, if exposure is sequential, steps of washing or rinsing the biomass after exposure to one (e.g. the first) oxidizing agent and prior to exposure to another (e.g. the second) may be carried out, with or without including a step of drying the biomass between exposures. Features of exemplary oxidizing and alkalinizing agents, and combinations thereof, are described below.

“Ozonation” or “ozonolysis” is the act of treating lignocellulosic biomass with ozone. The lignocellulosic biomass may be in an aqueous suspension or in a solid dry phase. In some aspects of the present invention, lignocellulosic biomass is initially contacted with a gas comprising ozone. Ozone oxidizes lignocellulosic polymers, causing delignification and fragmentation of the polymers and resulting in reduced hydrophobicity of the lignocellulosic materials, shorten time required for ammonia soaking and improved accessibility to saccharifying enzymes used in sugar production.

By a “gas comprising ozone” we mean a gas that is at least from about 80% to about 99% other gases, and is usually from about 1 to about 20% ozone. Other components of the gas may be any that can serve as a carrier for the ozone, including but not limited to “air”, for example compressed air; nitrogen; oxygen; carbon dioxide, carbon monoxide, hydrogen, helium etc.; and mixtures thereof. Such gaseous mixtures may be referred to herein as “ozone-enriched gas”.

The ozone-enriched gas is typically delivered to the biomass as a gaseous stream in an amount corresponding to a delivery rate in the range of from about 0.1 liters/minute to about 10 liters/minute or higher, and is generally in the range of from about 0.5 to about 5.0 L/min, e.g. 0.5, 1.0, 1.5, 2.0, 2.5, 3.0, 3.5, 4.0, 4.5, or 5.0 L/min. The gas may be blown onto or over the surface of the biomass, with the biomass preferably being agitated, stirred or mixed in some manner to incorporate the gas; or the gas may be bubbled into or through the biomass; or the gas may be introduced into a container (e.g. a closed container) that contains the biomass, with the gas becoming mixed with the biomass by rotation, shaking, or some other form of agitation, of the container; or the biomass and the gas may both flow contercurrently through a common channel or conduit, etc. Sufficient movement of the gas and the biomass with respect to each other is effected so as to provide contact between the ozone and the lignocellulosic material, with material becoming exposed to and/or suffused (infused, permeated, impregnated) with the gas in a manner that permits oxidation of the biomass to occur.

The ozone that is used may come from any suitable source. For example, ozone may be generated by methods such as the Corona discharge method, by ultraviolet (UV) ozone generators, by vacuum-ultraviolet (VUV) ozone generators, by cold plasma methods, by electrolytic ozone generation (FOG), etc. In some embodiments, ozone is generate from one or both of air and oxygen using e.g. an L11-L24 Ozone Generator manufactured by Pacific Ozone, Calif. USA.

The length of exposure to ozone is typically in the range of from about 2 minutes to about 2 hours, and is usually in the range of from about 2 minutes to about 60 minutes, e.g. about 2, 5, 10, 15, 20, 25, 30, 35, 40, 45, 50, 55 or 60 minutes or longer, e.g. 65, 70, 75, 80, 85, 90, 95, 100, 105, 110, 115, or 120 minutes. Ozone exposure is typically carried out at a pressure of from about 1 atm to about 1.5 atm or 1, 2, 3, 4, 5, or 6 psig.

In one aspect, the present method utilizes a combination of H₂O₂ and ozone (peroxone) to generate hydroxyl radicals to treat lignocellulosic biomass. The aspect employs the principle of the Fenton mechanism of highly reactive hydroxyl radicals generated from H₂O₂ for use in fragmenting lignin. Hydroxyl radicals are reactive towards degradation of aromatic lignin compounds, resulting into demethoxylation, β-O-4 ether linkage cleavage, hydroxylation and C_{α}-oxidation.

According to this aspect, sized biomass is exposed to or mixed with a solution of H₂O₂ (usually an aqueous solution) and then subsequently exposed to ozone as described above. The concentration of H₂O₂ that is added to the reaction is generally in the range of from about 0.25:1 to about 3:1 molar ratio of ozone, e.g. about 0.25:1, 0.5:1, 0.75:1, 1:1, 1.25:1, 1.5:1, 1.75:1, 2:1, 2.25:1, 2.5:1, 2.75:1, or 3:1. The amount of this concentrate that is added to the biomass is generally from about 100×10⁻⁶ to about 1000×10⁻⁶ (e.g. about 100, 150, 200, 250, 300, 350., 400, 450, 500, 550, 600, 650, 700, 750, 800, 850, 900, 950 or 1000×10⁻⁶) liters per gram of biomass, providing a final H₂O₂/biomass wt/wt ratio of from about 0.03 to about 0.3 (e.g. about 0.03, 0.05, 0.1, 0.15, 0.2, 0.25 or 0.3).

The mixture of biomass plus H₂O₂ (prepared in water) is placed in a vessel and exposed to ozone as discussed above, while under conditions of one or both of low temperature and low pressure. The length of exposure to ozone is typically in the range of from about 2 minutes to about 20 minutes (e.g. about 2, 3, 4, 5, 6, 7, 8, 9, 10, 11, 12, 13, 14, 15, 16, 17, 18, 19, or 20 minutes) and is usually in the range of from about 2 minutes to about 2 hours (e.g. about 10, 20, 30, 40, 50, 60, 70, 80, 90, 100, 110 or 120 minutes). Ozone exposure is typically carried out at a pressure of from about 1 atm to 1.5 atm or 1, 2, 3, 4, 5, or 6 psig.

“Combined Pretreatment” according to another aspect of the invention, ozonation or peroxone process is utilized as a rapid oxidizing agent and initial or preliminary lignin barrier remover, in order to increase the effectiveness and decrease the time required for pretreatment of the biomass with ammonia. The ammonia may be liquid or gaseous. Ozonation or peroxone initially alters the structure of lignin (Bule et al., 2013) and subsequent exposure to ammonia causes further changes (Gao et al., 2012) and reduces the crystalline structure of cellulose in the biomass which makes it a readily saccharifiable carbohydrate. If liquid (e.g. aqueous) ammonia is used, swelling of the biomass fibers also advantageously occurs.

The initial ozonation or peroxone treatment is typically carried out (as described above for ozone or peroxone pretreatment) for a period of time in the range of from about 2 min to about 60 min, and usually in the range of from about 2 min to about 2 hours. This step is carried out under conditions of low temperature and/or pressure, as described above. Thereafter, the treated biomass is either exposed to gaseous ammonia, or soaked in liquid ammonia, e.g. in a solution of aqueous ammonium hydroxide.

If gaseous ammonia is used, the treatment is carried out as follows: after ozonation or peroxone treatment biomass is to be packed in a cylindrical reactor and then gaseous ammonia is passed through the packed column for a desired period of time.

If liquid ammonia is used, the solution is generally aqueous and the concentration of ammonia in the solution is generally in the range of from about 5% to about 30% (e.g. about 5, 10, 15, 20, 25, or 30%). The ratio of biomass to aqueous ammonia is, for example, in the range of from about 5% to about 25% wt/wt (e.g. about 5, 10, 15, 20, or 25%).

The step of soaking in (exposing to, contacting with, etc.) aqueous ammonia is generally carried out for a period of time in the range of from about 30 minutes to about 24 hours, and usually in the range of from about 30 minutes to about several days. This step is also carried out under conditions of low temperature and/or pressure, e.g. at range of from about 20° C. to about 70° C. (e.g. about 20, 25, 30, 35, 40, 45, 50, 55, 60, 65, or 70° C.), and preferably from about 30° C. to about 60° C. Generally, a maximum temperature of about 60° C. is used in the practice of the invention.

“Hydrolytic” or “cellulolytic” enzymes refers to a collection of enzymes which will typically contain one or more cellulases, xylanases, or ligninases used to digest lignocellulosic biomass. Appropriate enzyme cocktail could include, for example, (1) one or more cellulases, (2) one or more hemicellulases, (3) one or more ligninases. Cellulases include endocellulase (endoglucanase), exocellulase (exoglucanase), and/or β-glucosidase (cellobiase) and the likes. Hemicellulases include β-xylosidase, α-L-arabinofuranosidase, xyloglucanase, acetyl xylan esterase, a-glucuronidase, endoxylanase, and the like. “Ligninase” includes lignin peroxidase, laccase, manganese peroxidase and the like.

“Hydrolysate” refers to a solution containing hydrolytic enzymes, pretreated lignocellulosic biomass, and sugars obtained from the action of the enzymes on the biomass.

“Enzyme consumption” is the amount of enzyme used to digest lignocellulosic biomass, and is considered using filter paper units (FPU) or cellobiose units (CBU) or grams of enzyme solution.

“Toxicants” or “inhibitors” are organic acid or furan compounds which inhibit growth of microorganisms e.g. acetic acid, furfural and 5-hydroxymethyl-2-furfural, etc.

“Saccharification” refers to the production of sugars from polysaccharides using hydrolytic enzymes.

“Oleaginous microorganism” or “oleaginous” when used to refer to a microorganism are any microbe (yeast, bacteria, fungi and algae) that is capable of producing lipid by fermenting sugar. Illustrative oleaginous yeast strains include: Cryptococcus curvatus, Rhodotorula glutinis, Rhodosporidium toruloides, Lipomyces starkeyi, Yarrowia lipolytica, Trichosporon fermentans and the likes. Exemplary are some of the potential lipid producing fungal strains, such as Rhizopus oryzae, Neosartorya fischeri, Chaetomium globosum, Aspergillus niger, Mortierella isabellina, Cunninghamella elegans, Mucor circinelloides, Aspergillus terreus, Umbelopsis vinacea, Mucor plumbeus and Thermomyces lanuginosus and the likes. Examples of bacteria that may be used in the practice of the invention include but are not limited to species such as mycobacteria, corynebacteria, nocardia, arthobacteria and the like. Algal species that may be used in the practice of the invention include but are not limited to include chlorella, dunaliella and the like.

“Fermentation” refers to the production of a target product using oleaginous microorganism, which feed on sugars produced from enzymatic hydrolysis.

“Waste products” are waste chemicals used in a pretreatment process. Waste chemicals include acids such as sulfuric acid or hydrochloric acid, and bases such as sodium hydroxide.

“Alkaline treatment” is the pretreatment of lignocellulosic biomass with a mixture of chemicals that is basic or high pH in nature.

“Room temperature” or “ambient” when used in reference to temperature refer to any temperature from between about 15° C. to about 30° C.

“Simultaneous saccharification and fermentation” (SSF) refers to a process in which the saccharification and fermentation steps take place in a single operation. In this process, the pretreated lignocellulosic biomass is mixed with saccharifying enzymes and oleaginous microorganism inoculums at once to undergo simultaneous saccharification and fermentation (SSF). The enzymatic cocktail breaks down complex polysaccharides in the di- or oligosaccharides or polysaccharides (e.g. cellobiose, xylobiose, glucotriose, cellulose, hemicellulose and a like) releasing simple sugars (e.g. glucose, xylose, mannose, arabinose, galactose) which the microorganisms use for growth and biofuel or bioproduct production. Solid and cellulase loadings may be varied as appropriate for particular conditions, e.g. type of microorganism employed, type of biomass, etc.

SSF is commonly applied in ethanol production. SSF is regarded as producing higher product yields and requiring lower amounts of enzyme, due to reduced end-product inhibition by cellobiose and glucose farmed during enzymatic hydrolysis (Elliston et al., 2013; Emert & Katzen, 1980; Emert et al., 1980; Gutierrez et al., 2013; Hoyer et al., 2013; Huang et al., 2013b; Spindler et al., 1989; Takagi et al., 1977). Combining SSF with hemicellulose sugar fermentation has attracted attention because of lower costs (Dien et al., 2000; Golias et al., 2002), and this combination may be utilized in the presets invention

“Physico-chemical factors” refers to the physical and chemical parameters of pretreatment, including but not limited to temperature, lignocellulosic biomass particle size, chemical usage and concentration, and residence time.

“Residence time” refers to the length of time in which the lignocellulosic biomass remains in the pretreatment reactor.

“Solid loading” refers to the proportion of solid to liquid in the pretreatment reactor.

“Aqueous ammonia” or “aqueous solution comprising ammonia” refers to the use of ammonia gas (NH₃) or ammonium ions (NH₄⁺) in water as ammonium hydroxide.

“Fragmentation” when used in reference to lignocellulosic biomass or lignin refers to the breakdown of structural bonds between lignin and lignin or lignin and carbohydrate molecules.

“Particle size” refers to the size of lignocellulosic biomass used in the pretreatment

“Mesh size” refers to the Tyler screen scale, which defines particle sizes by their ability to pass through a wire mesh.

“Treated” or “pretreated” when used in reference to biomass refers to biomass that has be subjected to a pretreatment.

“Moisture content” refers to the proportion of water contained within lignocellulosic biomass, or added to the lignocellulosic biomass.

“Solid residue” refers to solid biomass remaining after pretreatment and not in the aqueous form.

“Deactivated” when used in reference to enzymes refers to an enzyme that has been denatured by either heat or chemicals and is no longer functional.

“Microbial lipids” or “lipids” or “single cell oil” as used herein refers to lipids that can be biosynthesized and stored by oleaginous microorganisms such as bacteria, fungi, algae and yeasts using the methods of the invention. Such lipids include but are not limited to those which include one or more of the following fatty acids: myristic, palmitic, palmitoleic, stearic, oleic, linoleic, linolenic, and arachidonic acid. Particular lipids include but are not limited to: heptadecenoic acid, behenic acid, lignoceric acid, pentadecanoic acid, hexadecenoic acid, γ-linolenic acid and eicosenoic acid, etc.

The microbial lipids obtained using the processes described herein may be used for any suitable purpose such as fine and industrial chemicals. In one aspect, they are used to produce biofuel, particularly a “drop-in” biofuel. By “biofuel” we mean a fuel made from biologic materials. Prototypical biofuels that may be obtained using the lipids generated as described herein include but are not limited to renewable gasoline and diesel, jet fuel and biodiesel. One skilled in the art may be familiar with methods of extracting and processing lipids. After extraction, the oils may be used to form biofuel (e.g. methane, biodiesel, bioethanol and other alcohols, etc.) as described, illustrative are following example: US patents (Day et al., 2013; Oyler, 2011), the complete contents of which are hereby incorporated by reference.

A “drop-in” fuel is a fuel, usually a biofuel that does not require adaptation of conventional petroleum-based engine fuel systems or fuel distribution networks, prior to use. Drop-in fuel can be used “as is” and/or blended in any amount with other drop-in fuels, drop-in blends, and/or conventional fuels.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

Before exemplary embodiments of the present invention are described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

### EXAMPLES

The goal of the experimental work described below was to develop efficient, minimal toxicant producing, and economical pretreatment technology for processing lignocellulosic biomass (e.g. wheat straw, lawn grass) prior to enzymatic saccharification, in order to maximize production of monomeric sugars and minimize loss of such sugars. The present invention further details about utilization of produced monomeric sugars for biofuel and bioproduct production and are defined in the following Examples. It should be understood that these Examples, while indicating exemplary embodiments of the invention, are given by the way of illustration only.

The representative list enlists chemicals and materials used in the examples. All the analytical grade regents and chemical were used as received.

Glucose, xylose, cellobioase, mannose, galactose, citric acid, sodium hydroxide, ammonium hydroxide, hydrogen peroxide were obtained from Sigma-Aldrich (St. Louis, Mo.).

Wheat straw was obtained from the Grange Supply Co. in Pullman Wash. and hammer milled at the Washington State University's Wood Materials and Engineering Laboratory. The straw was then sieved through a particular mesh Tyler Standard Screen Scale. The resulting particles were then used for further pretreatment.

### Biomass Sugar Analysis

The biomass samples were dried at 105° C. in hot air oven for 24 h and then used for compositional analyses. NREL's standard laboratory analytical procedure (LAP) was utilized for carbohydrate and lignin content determination (Sluiter et al., 2004). In a two-stage acid hydrolysis procedure around 0.3 g of sample was weighed and treated. After initial hydrolysis at 37° C. with 3 mL of 72% (w/w) sulfuric acid, the samples were diluted with distilled water to a total volume of 84 mL and autoclaved for 1 h in pressure tubes. Sugars in the aqueous phase were determined using ion chromatography (Dionex ICS-3000 with Dionex Pac PA20 column and CarboPac PA20 guard column). The samples were run for 60 min, and the column was flushed between runs with 100% 200 mM NaOH followed by de-ionized water. Sugar concentration was calculated by comparison to a standard sugar sample, and all measurements were taken in triplicate.

### Saccharification or Enzymatic Hydrolysis

Enzymatic hydrolysis was performed on control and treated samples to estimate the sugar recovery before and after pretreatment. The hydrolysis was carried out at 1 to 20% solid loading in 50 mM sodium acetate buffer (pH 4.8) containing 100 μL 2% sodium azide, with 30 FPU (per 1 g biomass) of cellulase (Novozymes NS 50013) and 30 CBU (per 1 g biomass) of β-glycosidase (Novozymes NS 50010) at 50° C. for stipulated period of time in an orbital incubator shaker (Gyromax 747). In some of the embodiments, Cellic® CTec2 and HTec2 from Novozymes were utilized unless and otherwise stated. The total sugars released after stipulated period of time were used to calculate sugar recovery after enzymatic hydrolysis. The sugar composition of enzymatic hydrolysate was analyzed using DIONEX-IC as detailed in previous section (biomass sugar analysis).

### Example 1

### Sugar Analysis of Control, Ozone Treated, Soaking Aqueous Ammonia (SAA) Treated, and Combined (Ozone Followed by SAA Treated Hence Forward OSAA) Wheat Straw

The sugar analysis of control, ozone treated sample for 2 hour (conditions: (ozonation: 90% moisture adjustment of 42-60 mesh particles, 2 hour reaction time with 5.4% ozone in 2 L/minute oxygen gas flow rate), only SAA treated (conditions 60° C. and 24 hours), OSAA treated (ozonation: 90% moisture adjustment of 42-60 mesh particles, 10 min reaction time with 5.4% ozone in 2 L/minute oxygen gas flow rate; SAA: 20% solid loading (w/v), 20% ammonium hydroxide in water (w/v), 50° C. reaction temperature and 6 hours residence time) of wheat straw samples are shown in Table 1. The concentration of monosaccharide for ozone treated samples decreased minutely, which could be reaction of ozone with sugars. The representative sugar concentration of SAA and OSAA treated samples increased significantly when measured on percentage basis, which is collective effect of removal of extractives, ash and lignin to the extent.

TABLE 1

| Components | Control | Ozone | SAA | OSAA |
|---|---|---|---|---|
| Arabinose | 3.15 | 2.79 | 2.96 | 1.97 |
| Galactose | 0.55 | 0.47 | 0.56 | 0.32 |
| Glucose | 36.19 | 36.03 | 37.75 | 53.86 |
| Xylose/Mannose | 16.72 | 14.02 | 15.82 | 22.67 |
| Total sugars | 56.61 | 53.31 | 57.09 | 78.82 |

Note:
All sugar concentration mentioned in this table represents % sugar present

### Example 2

### Effect of Pretreating Wheat Straw Particles with Ozone Alone on Sugar Recovery

Wheat straw particles (42-60 mesh size) were adjusted with 90% (w/w) moisture content and treated with stream of ozone-enriched oxygen gas containing 5.4% ozone (oxygen flow rate 2 L/min) at room temperature (23° C.) in stainless steel reactor for 5 to 30 min. The treated biomass was removed and saccharified according to the following procedure. The wheat straw residue from the above procedure (0.2 g) was added to 20 ml citrate buffer (pH=4.8), Celluclas® 1.5 L (71.0 μL, protein concentration=126.5 mg/mL) and Novozym 188 (14.0 μL, protein concentration=134 mg/mL) enzyme mixture, and the mixture was left stirring in an incubator shaker at 50° C. Samples were removed after 72 hours, enzyme was deactivated by boiling for 5 min and sugar concentration was analyzed by DIONEX-ion chromatography. Results are shown in Table 2. The results showed that pretreating wheat straw particles with ozone increases sugar recovery as opposed to control sample.

TABLE 2

| Ozone | | | | | |
|---|---|---|---|---|---|
| treatment | | | | | |
| time | Arabinose^{§} | Galactose^{§} | Glucose^{§} | Xylose^{§} | Total sugar^{§} |
| 0 | 9.42 | 0.00 | 14.37 | 3.83 | 9.85 |
| 5 | 26.22 | 19.81 | 43.83 | 25.09 | 35.46 |
| 10 | 30.09 | 24.41 | 56.56 | 32.32 | 45.58 |
| 15 | 33.95 | 22.63 | 58.60 | 37.77 | 49.04 |
| 20 | 31.35 | 22.16 | 62.30 | 36.21 | 50.35 |
| 25 | 30.43 | 22.86 | 65.82 | 38.57 | 53.22 |
| 30 | 31.38 | 23.98 | 67.18 | 40.03 | 54.60 |

Note:
^{§}sign represents % sugar recovery

### Example 3

### Effect of Peroxone Treatment on Enzymatic Hydrolysis of Wheat Straw Particles

Wheat straw particles (42-60 mesh size) 3 g were thoroughly mixed with water containing H₂O₂ (concentration was adjusted to deliver 1:2 molar ratio of H₂O₂ to ozone) to adjust 90% (w/w) moisture content. This mixture was placed in stainless steel reactor and treated with stream of ozone-enriched oxygen gas containing 5.4% ozone (oxygen flow rate 2 L/min) to generate highly reacting hydroxyl radical at room temperature (23° C.) for 5 to 30 min. The treated biomass was removed and saccharified according to the following procedure. The wheat straw residue from the above procedure (0.2 g) was added to 20 ml citrate buffer (pH=4.8), Celluclast® 1.5 L (71.0 μL, protein concentration=126.5 mg/mL) and Novozym 188 (14.0 μL, protein concentration=134 mg/mL) enzyme mixture, and the mixture was left stirring in an incubator shaker at 50° C. Samples were removed after 72 hours, enzyme was deactivated by boiling for 5 min and sugar concentration was analyzed by DIONEX-ion chromatography. The results are shown in Table 3. The results showed that pretreating wheat straw particles with peroxone further increases sugar recovery as opposed to Example 2 and control sample.

TABLE 3

| Peroxone | | | | | |
|---|---|---|---|---|---|
| treatment | | | | | |
| time | Arabinose^{§} | Galactose^{§} | Glucose^{§} | Xylose^{§} | Total sugar^{§} |
| 0 | 9.42 | 0.00 | 14.37 | 3.83 | 9.85 |
| 5 | 35.57 | 21.55 | 48.58 | 27.19 | 39.30 |
| 10 | 34.62 | 20.81 | 59.64 | 32.89 | 47.67 |
| 15 | 45.02 | 20.74 | 63.28 | 38.14 | 52.19 |
| 20 | 51.12 | 23.91 | 74.74 | 44.67 | 61.41 |
| 25 | 48.17 | 22.13 | 77.65 | 40.66 | 61.28 |
| 30 | 39.22 | 29.98 | 72.95 | 50.03 | 62.15 |

Note:
^{§}sign represents % sugar recovery

### Examples 4 to 12

### Effect of Combined Pretreatment of Ozone and Soaking Aqueous Ammonia (SAA)

Wheat straw particles (42-60 mesh size) were adjusted with 90% (w/w) moisture content and treated with stream of ozone-enriched oxygen gas containing 5.4% ozone (oxygen flow rate 2 L/min) at room temperature (23° C.) in stainless steel reactor for 10, 15 and 20 minutes. Each of the ozone pretreated particles were treated subsequently using 20% (w/w) aqueous ammonium hydroxide solution for 3, 6 and 9 h at 50° C. at 20% (w/v) solid loading. Resultant solid residue was filtered, separated and washed with distilled water until neutral pH achieved. The washed solid residue was dried at 50° C. to generate pretreated biomass. The wheat straw residue from the above procedure (0.2 g) was added to 20 ml citrate buffer (pH=4.8), Celluclast® 1.5 L (71.0 μL, protein concentration=126.5 mg/mL) and Novozym 188 (14.0 μL, protein concentration=134 mg/mL) enzyme mixture, and the mixture was left stirring in an incubator shaker at 50° C. Samples were removed after 12, 24, 48 and 72 hours, enzyme was deactivated by boiling for 5 min and sugar concentration was analyzed by DIONEX-ion chromatography. The results are shown in Table 4. The results showed that combined pretreatment methods are very efficient for sugar recovery, either ozonation or peroxone treatment prior to SAA pretreatment will be economical process for obtaining cheap sugars from lignocellulosic biomass.

TABLE 4

| Combined | Enzymatic | | | | |
|---|---|---|---|---|---|
| treatment | hydrolysis | | | | |
| condition | time (hours) | Arabinose^{§} | Galactose^{§} | Glucose^{§} | Xylose |
| Example 4: 10 minutes | 12 | 36.15 | 34.28 | 83.24 | 77.03 |
| ozonation + 3 hour SAA | 24 | 37.27 | 35.30 | 91.43 | 78.09 |
| | 48 | 62.83 | 42.40 | 93.89 | 93.07 |
| | 72 | 70.85 | 49.97 | 98.64 | 99.34 |
| Example 5: 10 minutes | 12 | 34.71 | 34.82 | 87.49 | 77.99 |
| ozonation + 6 hour SAA | 24 | 44.12 | 34.82 | 97.46 | 84.68 |
| | 48 | 69.27 | 39.07 | 93.46 | 97.42 |
| | 72 | 83.05 | 45.45 | 105.23 | 98.45 |
| Example 6: 10 minutes | 12 | 36.29 | 42.35 | 92.77 | 86.82 |
| ozonation + 9 hour SAA | 24 | 43.64 | 42.35 | 96.29 | 88.83 |
| | 48 | 60.26 | 48.59 | 101.56 | 90.78 |
| | 72 | 81.64 | 49.26 | 105.28 | 98.17 |
| Example 7: 15 minutes | 12 | 38.28 | 43.46 | 73.73 | 71.48 |
| ozonation + 3 hour SAA | 24 | 45.75 | 48.55 | 89.80 | 82.13 |
| | 48 | 72.20 | 48.55 | 91.02 | 96.10 |
| | 72 | 78.98 | 50.32 | 99.42 | 98.47 |
| Example 8: 15 minutes | 12 | 41.02 | 42.97 | 80.12 | 77.16 |
| ozonation + 6 hour SAA | 24 | 46.44 | 45.23 | 88.11 | 79.51 |
| | 48 | 69.48 | 47.97 | 88.09 | 93.44 |
| | 72 | 76.55 | 48.10 | 98.29 | 98.97 |
| Example 9: 15 minutes | 12 | 39.50 | 34.82 | 76.63 | 76.81 |
| ozonation + 9 hour SAA | 24 | 46.76 | 49.97 | 89.79 | 83.13 |
| | 48 | 61.55 | 24.99 | 88.81 | 91.27 |
| | 72 | 91.27 | 49.97 | 99.91 | 98.43 |
| Example 10: 20 minutes | 12 | 31.36 | 34.35 | 77.64 | 70.78 |
| ozonation + 3 hour SAA | 24 | 37.35 | 34.82 | 87.75 | 75.12 |
| | 48 | 62.96 | 42.40 | 95.99 | 93.97 |
| | 72 | 78.18 | 43.48 | 104.41 | 95.77 |
| Example 11: 20 minutes | 12 | 35.42 | 38.55 | 78.03 | 83.06 |
| ozonation + 6 hour SAA | 24 | 42.86 | 34.51 | 79.26 | 85.20 |
| | 48 | 56.00 | 38.59 | 92.39 | 89.37 |
| | 72 | 81.43 | 42.65 | 101.67 | 97.84 |
| Example 12: 20 minutes | 12 | 34.27 | 32.10 | 67.89 | 65.71 |
| ozonation + 9 hour SAA | 24 | 46.01 | 33.55 | 86.97 | 83.44 |
| | 48 | 58.26 | 34.45 | 92.03 | 90.56 |
| | 72 | 73.12 | 35.53 | 101.20 | 96.59 |

Note:
§ sign represents % sugar recovery

### Example 13 to 24

### Effect of High Solid Loading and Different Enzyme Loading on Sugar Release of OSAA Pretreated Wheat Straw

Two sets of experiments were performed with wheat straw particles (42-60 mesh) pretreated using methods explained in Example 4 and with control (untreated) sample. For the examples 13 to 18, the wheat straw residue from the above methods was added to 20 ml citrate buffer (pH=4.8) at 5%, 7.5% and 10% w/v level, with addition of 30 FPU Celluclast® 1.5 L and 30 CBU Novozym 188 enzyme mixture, and the mixture was left stirring in an incubator shaker at 50° C. Samples were removed after 1, 3, 6, 12, 24, 36, 48 and 72 hours, enzyme was deactivated by boiling for 5 min and sugar concentration was analyzed by DIONEX-ion chromatography. Examples 12, 13, and 14 represent control samples while examples 15, 16 and 17 represents OSAA pretreated samples. The results of the study are shown in Table 5.

Similarly, for the examples 19 to 24, the wheat straw residue from the Examples 3 was utilized in this study. Different solid loading used for experiments was 5%, 7.5% and 10% w/v in 20 ml citrate buffer (pH=4.8) with 15 FPU Celluclast® 1.5 L and 15 CBU Novozym 188 enzyme mixture, and the mixture was left stirring in an incubator shaker at 50° C. Samples were removed after 1, 3, 6, 12, 24, 36, 48 and 72 hours, enzyme was deactivated by boiling for 5 min and sugar concentration was analyzed by DIONEX-ion chromatography. Examples 18, 19, and 20 represents control samples while examples 21, 22 and 23 represents OSAA pretreated samples. The results of the study are shown in Table 6. The results in Table 5 and 6 showed that the lignocellulosic biomass treated using OSAA pretreatment appropriate for producing concentrated single sugar stream of C5 and C6 sugars.

TABLE 5

| Time | Exam- | Exam- | Exam- | Exam- | Exam- | Exam- |
|---|---|---|---|---|---|---|
| in hour | ple 13 | ple 14 | ple 15 | ple 16 | ple 17 | ple 18 |
| 1 | 5.97 | 5.93 | 6.15 | 30.27 | 24.43 | 23.63 |
| 3 | 6.56 | 7.56 | 7.13 | 41.36 | 33.72 | 30.89 |
| 6 | 7.88 | 8.42 | 8.24 | 65.58 | 63.86 | 59.88 |
| 12 | 8.84 | 11.59 | 9.84 | 70.60 | 67.52 | 61.92 |
| 24 | 12.82 | 14.83 | 13.19 | 74.15 | 70.18 | 67.41 |
| 36 | 13.78 | 16.21 | 13.95 | 83.09 | 76.07 | 75.03 |
| 48 | 17.72 | 18.40 | 15.63 | 88.23 | 81.22 | 78.54 |
| 72 | 19.26 | 20.58 | 17.31 | 96.90 | 92.70 | 91.34 |

Note:
Example 13: control sample with 5% (w/v) solid loading and 30 CBU and 30 FPU enzyme loading; Example 14: control sample with 7.5% (w/v) solid loading and 30 CBU and 30 FPU enzyme loading; Example 15: control sample with 10% (w/v) solid loading and 30 CBU and 30 FPU enzyme loading; Example 16: OSAA treated sample with 5% (w/v) solid loading and 30 CBU and 30 FPU enzyme loading; Example 17: OSAA treated sample with 7.5% (w/v) solid loading and 30 CBU and 30 FPU enzyme loading; Example 18: OSAA treated sample with 10% (w/v) solid loading and 30 CBU and 30 FPU enzyme loading; and all the sugar recovery results mentioned here are in percentage.

TABLE 6

| Time | Exam- | Exam- | Exam- | Exam- | Exam- | Exam- |
|---|---|---|---|---|---|---|
| in hour | ple 19 | ple 20 | ple 21 | ple 22 | ple 23 | ple 24 |
| 1 | 5.38 | 4.98 | 4.34 | 28.57 | 20.61 | 20.47 |
| 3 | 6.77 | 6.35 | 5.67 | 31.05 | 30.41 | 23.65 |
| 6 | 7.54 | 6.53 | 6.09 | 49.35 | 55.10 | 48.33 |
| 12 | 9.25 | 9.84 | 9.23 | 60.99 | 60.06 | 55.50 |
| 24 | 16.35 | 15.22 | 12.35 | 65.92 | 63.71 | 64.63 |
| 36 | 16.38 | 15.77 | 13.41 | 82.89 | 78.58 | 75.09 |
| 48 | 18.82 | 18.17 | 15.36 | 86.18 | 82.63 | 86.20 |
| 72 | 21.27 | 20.56 | 17.32 | 90.90 | 88.69 | 91.97 |

Note:
Example 19: control sample with 5% (w/v) solid loading and 15 CBU and 15 FPU enzyme loading; Example 20: control sample with 7.5% (w/v) solid loading and 15 CBU and 15 FPU enzyme loading; Example 21: control sample with 10% (w/v) solid loading and 15 CBU and 15 FPU enzyme loading; Example 22: OSAA treated sample with 5% (w/v) solid loading and 15 CBU and 15 FPU enzyme loading; Example 23: OSAA treated sample with 7.5% (w/v) solid loading and 15 CBU and 15 FPU enzyme loading; Example 24: OSAA treated sample with 10% (w/v) solid loading and 15 CBU and 15 FPU enzyme loading; and all the sugar recovery results mentioned here are in percentage.

### Example 25

### Utilization of Single Stream of C5 and C6 Sugars for Microbial Lipid Production

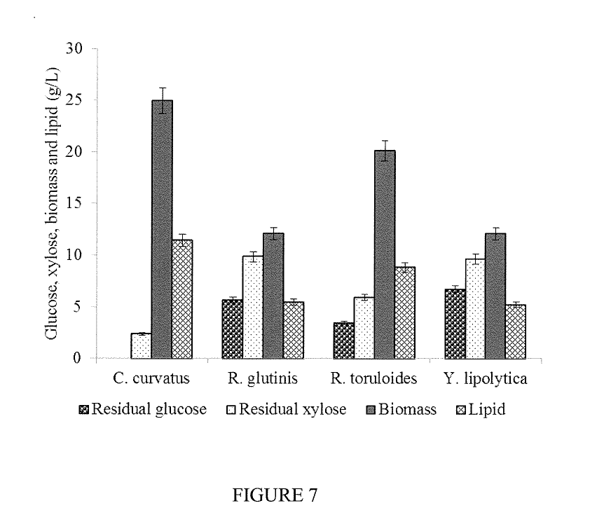

Oleaginous yeast strains Cryptococcus curvatus, Rhodotorula glutini, Rhodosporidium toruloides, Yarrowia lipolytica were grown on mixture of C5 and C6 sugars produced during enzymatic hydrolysis process described in example 23. Initially, seed culture of all the strains were produced with YPD medium containing nutritional components such as yeast extract 10 g/L, peptone 10 g/L, and glucose 20 g/L and incubating it at conditions of temperature 30° C., shaking at 150 rpm for 24 hours. About 10% of seed culture was added to the production medium containing enzymatic hydrolysate 50 ml, ammonium sulphate 0.5 g/L, yeast extract 1.0 g/L, potassium dihydrogen phosphate 0.4 g/L, magnesium sulphate 1.5 g/L with pH adjustment to 6.0. The cultures were maintained at 28° C. and 200 rpm in 250 Erlenmeyer flask. The cultures were incubated for 4 days and cell were harvested to determine the accumulated lipid content by lipid analysis method reported by O'Fallon et al., (2007). The results of the study are shown in the FIG. 4. It was observed that single stream of C5 and C6 sugars are suitable for growing oleaginous yeasts. As FIG. 7 shows, C. curvatus was the highest biomass as well as lipid accumulating strain about 24.97 g/L and 11.43 g/L respectively, as compared to the other strains used in present invention. C. curvatus utilized all of the C6 sugars (glucose) while leaving only trace amount of C5 (xylose sugars).

### Example 26

### Simultaneous Saccharification and Fermentation (SSF) of OSAA Treated Wheat Straw Particles by Oleaginous Yeast for Microbial Oil Production

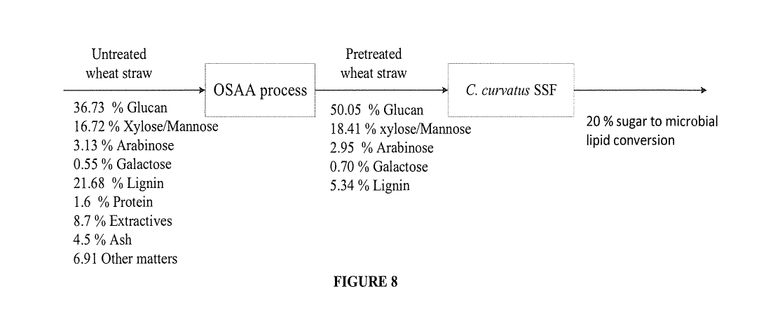

The pretreated biomass obtained after one of the examples 3 to 11 was utilized in this example. Culture of C. curvatus oleaginous yeast was exploited during this investigation. The seed culture preparation and supplementation of other nutritional component was similar as that of example 24. The experiment performed at 7.5% (w/v) solid loading, 30 FPU and 30 CBU enzyme loading at 30° C., shaking at 200 rpm and incubating for 4 days shows around 20% direct sugar to microbial lipid conversion. FIG. 8 shows mass balance of processing 100 gm of biomass using OSAA pretreatment followed by simultaneous saccharification and fermentation of C. curvatus. The pretreated biomass obtained after OSAA pretreatment methods reported in this invention is particularly suitable for microbial lipid production as other methods like dilute acid pretreatment, ammonia fiber explosion (AFEX), steam explosion, and some of the pretreatment methods mentioned in U.S. No. 20100159521A1 (Cirakovic & Diner, 2010b) which retains maximum of lignin in the biomass.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

### REFERENCES

Akhtar, P., Gray, J. I., Asghar, A. 1998. Synthesis of lipids by certain yeast strains grown on whey permeate. Journal of Food Lipids, 5(4), 283-297.
Alvarez, R., Rodriguez, B., Romano, J., Diaz, A., Gomez, E., Miro, D., Navarro, L., Saura, G., Garcia, J. 1992. Lipid accumulation in Rhodotorula glutinis on sugar cane molasses in single-stage continuous culture. World Journal of Microbiology and Biotechnology, 8(2), 214-215.
Angerbauer, C., Siebenhofer, M., Mittelbach, M., Guebitz, G. 2008. Conversion of sewage sludge into lipids by Lipomyces starkeyi for biodiesel production. Bioresource Technology, 99(8), 3051-3056.
Ben-Ghedalia, D., Shefet, G., Miron, J. 1980. Effect of ozone and ammonium hydroxide treatments on the composition and in-vitro digestibility of cotton straw. Journal of the Science of Food and Agriculture, 31(12), 1337-1342.
Bule, M., Gao, A., Hiscox, B., Chen, S. 2013. Structural modification of lignin and characterization of pretreated wheat straw by ozonation. Journal of Agricultural and Food Chemistry, 61 (16), 3916-3925.
Cardona, C. A., Sanchez, Ó. J. 2007. Fuel ethanol production: process design trends and integration opportunities. Bioresource Technology, 98(12), 2415-2457.
Carere, C. R., Sparling, R., Cicek, N., Levin, D. B. 2008. Third generation biofuels via direct cellulose fermentation. International Journal of Molecular Sciences, 9(7), 1342-60.
Chandra, R. P., Bura, R., Mabee, W. E., Berlin, A., Pan, X., Saddler, J. N. 2007. Substrate pretreatment: the key to effective enzymatic hydrolysis of lignocellulosics? Advances in Biochemical Engineering/Biotechnology, 108, 67-93.
Chen, X., Li, Z., Zhang, X., Hu, F., Ryu, D. D. Y., Bao, J. 2009. Screening of oleaginous yeast strains tolerant to lignocellulose degradation compounds. Applied Biochemistry and Biotechnology, 159(3), 591-604.
Cirakovic, J., Diner, A. B. 2010a. Organosolv and ozone treatment of biomass to enhance enzymatic saccharification. US 2010015922A1.
Cirakovic, J., Diner, B. A. 2010b. Ozone treatment of biomass to enhance enzymatic saccharification. US 2010/0159521A1.
Dale, B. E. 2012. Process for the treatment of lignocellulosic biomass. US 20,120,325,202.
Day, A. G., Brooks, G., Franklin, S. 2013. Modified lipids produced from oil-bearing microbial biomass and oils. US 20,130,005,005.
Demirba [symbol] , A. 2003. Energy and environmental issues relating to greenhouse gas emissions in Turkey. Energy Conversion and Management, 44(1), 203-213.
Dien, B. S., Nichols, N. N., O'Bryan, P. J., Bothast, R. J. 2000. Development of new ethanologenic Escherichia coli strains for fermentation of lignocellulosic biomass. Applied Biochemistry and Biotechnology, 84-86, 181-196.
Duarte, S. H., Andrade, C. C. P. d., Ghiselli, G., Maugeri, F. 2013. Exploration of brazilian biodiversity and selection of a new oleaginous yeast strain cultivated in raw glycerol. Bioresource Technology, 138, 377-381.
Elliston, A., Collins, S. R. A., Wilson, D. R., Roberts, I. N., Waldron, K. W. 2013. High concentrations of cellulosic ethanol achieved by fed batch semi simultaneous saccharification and fermentation of waste-paper. Bioresource Technology, 134(0), 117-126.
Emert, G. H., Katzen, R. 1980. Gulfs cellulose-to-ethanol process. Chemtech, October, 610-614.
Emert, G. H., Katzen, R., Fredrickson, R. E., Kaupisch, K. F. 1980. Gasohol/biomass developments: Economic update of the Gulf cellulose alcohol process. Chemical Engineering Progress, September, 47-52.
Gao, A. H., Bule, M. V., Laskar, D. D., Chen, S. 2012. Structural and thermal characterization of wheat straw pretreated with aqueous ammonia soaking. Journal of Agricultural and Food Chemistry, 60(35), 8632-8639.
Gao, D., Zeng, J., Zheng, Y., Yu, X., Chen, S. 2013. Microbial lipid production from xylose by Mortierella isabellina. Bioresource Technology, 133(0), 315-321.
Garcia-Cubero, M. A., Gonzalez-Benito, G., Indacoechea, I., Coca, M., Bolado, S. 2009. Effect of ozonolysis pretreatment on enzymatic digestibility of wheat and rye straw. Bioresource technology, 100(4), 1608-13.
García-Cubero, M. T., Palacín, L. G., González-Benito, G., Bolado, S., Lucas, S., Coca, M. 2012. An analysis of lignin removal in a fixed bed reactor by reaction of cereal straws with ozone. Bioresource technology, 107(0), 229-234.
Golias, H., Dumsday, G. J., Stanley, G. A., Pamment, N. B. 2002. Evaluation of a recombinant Klebsiella oxytoca strain for ethanol production from cellulose by simultaneous saccharification and fermentation: comparison with native cellobiose-utilising yeast strains and performance in co-culture with thermotolerant yeast and Zymomonas mobilis. Journal of Biotechnology, 96(2), 155-168.
Gonzalez-Garcia, Y., Hernandez, R., Zhang, G., Escalante, F. M., Holmes, W., French, W. T. 2013. Lipids accumulation in Rhodotorula glutinis and Cryptococcus curvatus growing on distillery wastewater as culture medium. Environmental Progress and Sustainable Energy, 32(1), 69-74.
Gutierrez, C., Mitchinson, C., Huang, T. T., Diner, B. A., Fagan, P. J., Hitz, W. D. 2013. Methods for improving the efficiency of simultaneous saccharification and fermentation reactions. US 20,130,143,277.
Himmel, M. E., Ding, S.-Y., Johnson, D. K., Adney, W. S., Nimlos, M. R., Brady, J. W., Foust, T. D. 2007. Biomass recalcitrance: engineering plants and enzymes for biofuels production. Science, 315(5813), 804-807.
Hoyer, K., Galbe, M., Zacchi, G. 2013. The effect of prehydrolysis and improved mixing on high-solids batch simultaneous saccharification and fermentation of spruce to ethanol. Process Biochemistry, 48(2), 289-293.
Huang, C., Chen, X.-f., Xiong, L., Yang, X.-y., Chen, X.-d., Ma, L.-l., Chen, Y. 2013a. Microbial oil production from corncob acid hydrolysate by oleaginous yeast Trichosporon coremiiforme. Biomass and Bioenergy, 49, 273-278.
Huang, C., Zong, M.-h., Wu, H., Liu, Q.-p. 2009. Microbial oil production from rice straw hydrolysate by Trichosporon fermentans. Bioresource Technology, 100(19), 4535-4538.
Huang, Y., Jin, Y., Fang, Y., Li, Y., Zhang, G., Xiao, Y., Chen, Q., Zhao, H. 2013b. Simultaneous saccharification and fermentation (SSF) of non-starch polysaccharides and starch from fresh tuber of Canna edulis ker at a high solid content for ethanol production. Biomass and Bioenergy, 52, 8-14.
Ishibashi, T., Ishida, M., Odawara, Y. 1985. Method for pretreatment of cellulose materials. EP 0,045,500.
Jeoh, T., Ishizawa, C. I., Davis, M. F., Himmel, M. E., Adney, W. S., Johnson, D. K. 2007. Cellulase digestibility of pretreated biomass is limited by cellulose accessibility. Biotechnology and Bioengineering, 98(1), 112-22.
Joseph, J., Pikulin, M. A., Friend, W. H. 2001. Process for conditioning ozone gas recycle stream in ozone pulp bleaching. U.S. Pat. No. 6,126,781,
Kim, T. H., Lee, Y. 2005. Pretreatment of corn stover by soaking in aqueous ammonia. Twenty-Sixth Symposium on Biotechnology for Fuels and Chemicals. Springer. pp. 1119-1131.
Kim, T. H., Lee, Y. 2007. Pretreatment of corn stover by soaking in aqueous ammonia at moderate temperatures. Applied Biochemistry and Biotechnology, 137(1-12), 81-92.
Kim, T. H., Taylor, F., Hicks, K. B. 2008. Bioethanol production from barley hull using SAA (soaking in aqueous ammonia) pretreatment. Bioresource Technology, 99(13), 5694-5702.
Kitcha, S., Cheirsilp, B. 2013. Enhancing lipid production from crude glycerol by newly isolated oleaginous yeasts: strain selection, process optimization, and fed-batch strategy. BioEnergy Research, 6(1), 300-310.
Lim, W.-S., Kim, J.-Y., Kim, H.-Y., Choi, J.-W., Choi, I.-G., Lee, J.-W. 2013. Structural properties of pretreated biomass from different acid pretreatments and their effects on simultaneous saccharification and ethanol fermentation. Bioresource Technology, 139(0), 214-219.
Lin, Y.-S., Lee, W.-C., Duan, K.-J., Lin, Y.-H. 2013. Ethanol production by simultaneous saccharification and fermentation in rotary drum reactor using thermotolerant Kluveromyces marxianus. Applied Energy, 105(0), 389-394.
Lynd, L. R., Wyman, C. E., Gerngross, T. U. 1999. Biocommodity engineering. Biotechnology Progress, 15(5), 777-793.
Meesters, P. A. E. P., Huijberts, G. N. M., Eggink, G. 1996. High cell density cultivation of the lipid accumulation yeast Cryptococcus curvatus using glycerol as a carbon source. Applied Microbiology and Biotechnology, 45(5), 575-579.
Mosier, N., Wyman, C., Dale, B., Elander, R., Lee, Y. Y., Holtzapple, M., Ladisch, M. 2005. Features of promising technologies for pretreatment of lignocellulosic biomass. Bioresource Technology, 96(6), 673-86.
Nothe, T., Fahlenkamp, H., von Sonntag, C. 2009. Ozonation of wastewater: rate of ozone consumption and hydroxyl radical yield. Environmental Science and Technology, 43(15), 5990-5.
O'fallon, J., Busboom, J., Nelson, M., Gaskins, C. 2007. A direct method for fatty acid methyl ester synthesis: application to wet meat tissues, oils, and feedstuffs. Journal of Animal Science, 85(6), 1511-1521.
Oyler, J. R. 2011. Two-stage process for producing oil from microalgae. U.S. Pat. No. 7,905,930.
Papanikolaou, S., Aggelis, G. 2002. Lipid production by Yarrowia lipolytica growing on industrial glycerol in a single-stage continuous culture. Bioresource Technology, 82(1), 43-49.
Peng, W.-f., Huang, C., Chen, X.-f., Xiong, L., Chen, X.-d., Chen, Y., Ma, L.-l. 2013. Microbial conversion of wastewater from butanol fermentation to microbial oil by oleaginous yeast Trichosporon dermatis. Renewable Energy, 55, 31-34.
Schneider, T., Graeff-Hönninger, S., French, W., Hernandez, R., Merkt, N., Claupein, W., Hetrick, M., Pham, P. 2013. Lipid and carotenoid production by oleaginous red yeast Rhodotorula glutinis cultivated on brewery effluents. Energy, in press.
Sluiter, A., Hames, B., Ruiz, R., Scarlata, C., Sluiter, J., Templeton, D., Crocker, D. 2004. Determination of structural carbohydrates and lignin in biomass. NREL Laboratory Analytical Procedure, Golden, Co.
Spindler, D. D., Wyman, C. E., Grohmann, K. 1989. Evaluation of thermotolerant yeasts in controlled simultaneous saccharifications and fermentations of cellulose to ethanol. Biotechnology and Bioengineering, 34(2), 189-195.
Suriyachai, N., Weerasaia, K., Laosiripojana, N., Champreda, V., Unrean, P. 2013. Optimized simultaneous saccharification and co-fermentation of rice straw for ethanol production by Saccharomyces cerevisiae and Scheffersomyces stipitis co-culture using design of experiments. Bioresource Technology, 142(0), 171-178.
Takagi, M., Abe, S., Suzuki, S., Emert, G. H., Yata, N. 1977. A method for production of alcohol directly from cellulose using cellulase and yeast. Proceedings, Bioconversion Symposium, Delhi, India. Indian Institute of Technology. pp. 551-571.
Tao, L., Aden, A., Elander, R. T., Pallapolu, V. R., Lee, Y. Y., Garlock, R. J., Balan, V., Dale, B. E., Kim, Y., Mosier, N. S., Ladisch, M. R., Falls, M., Holtzapple, M. T., Sierra, R., Shi, J., Ebrik, M. A., Redmond, T., Yang, B., Wyman, C. E., Hames, B., Thomas, S., Warner, R. E. 2011. Process and technoeconomic analysis of leading pretreatment technologies for lignocellulosic ethanol production using switchgrass. Bioresource Technology, 102(24), 11105-14.
Tsigie, Y. A., Wang, C.-Y., Truong, C.-T., Ju, Y.-H. 2011. Lipid production from Yarrowia lipolytica Polg grown in sugarcane bagasse hydrolysate. Bioresource Technology, 102(19), 9216-9222.
Ykema, A., Verbree, E. C., Kater, M. M., Smit, H. 1988. Optimization of lipid production in the oleaginous yeast Apiotrichum curvatum, in whey permeate. Applied Microbiology and Biotechnology, 29(2-3), 211-18.
Yoo, C. G., Nghiem, N. P., Hicks, K. B., Kim, T. H. 2013. Maximum production of fermentable sugars from barley straw using optimized soaking in aqueous ammonia (SAA) pretreatment. Applied Biochemistry and Biotechnology, 1-12.
Yu, X., Zheng, Y., Dorgan, K. M., Chen, S. 2011. Oil production by oleaginous yeasts using the hydrolysate from pretreatment of wheat straw with dilute sulfuric acid. Bioresource Technology, 102(10), 6134-6140.
Zeng, J., Zheng, Y., Yu, X., Yu, L., Gao, D., Chen, S. 2013. Lignocellulosic biomass as a carbohydrate source for lipid production by Mortierella isabellina. Bioresource Technology, 128(0), 385-391.
Zhao, X., Hu, C., Wu, S., Shen, H., Zhao, Z. 2010. Lipid production by Rhodosporidium toruloides Y4 using different substrate feeding strategies. Journal of Industrial Microbiology and Biotechnology, 1-6.

We claim:

1. A method of pretreating lignocellulosic biomass prior to enzymatic hydrolytic saccharification, comprising
mixing said biomass with an H₂O₂ solution prepared in water to form 0% to 90% (w/w) moisture content of biomass and
exposing said biomass/H₂O₂ mixture to ozone gas, wherein said step of exposing is carried out at a temperature of 15° C. to 30° C.

2. The method of claim 1, wherein said method includes a step of sizing said biomass prior to said step of mixing.

3. The method of claim 1, wherein said step of exposing is carried out for a time period ranging from about 1 to about 20 minutes.

4. The method of claim 3, wherein said step of exposing is carried out for at least about 15 minutes.

5. The method of claim 1, wherein said step of exposing is followed by one or both of a step of rinsing and a step of drying, prior to said enzymatic hydrolytic saccharification.

6. The method of claim 1, wherein sugars obtained from said enzymatic hydrolytic saccharification are used for lipid production by oleaginous microorganisms.

7. A method of pretreating biomass prior to enzymatic hydrolytic saccharification, comprising
exposing said biomass to ozone at a temperature of 15° C. to 30° C., and
contacting ozone-exposed biomass with aqueous ammonia at a temperature of or below about 50° C. and pressure lower than 1 atm.

8. The method of claim 7, wherein said method includes a step of sizing said biomass prior to said step of exposing.

9. The method of claim 7, wherein said step of contacting is carried out for a time period of from about 3 to about 36 hours.

10. The method of claim 9, wherein said step of contacting is carried out for from about 6 to about 9 hours.

11. The method of claim 7, wherein said second step of contacting is carried out below 1 atmp.

12. The method of claim 7, wherein the produced biomass with lower lignin concentration is particularly suitable for simultaneous saccharification and fermentation process of any biofuel or bioproduct.

13. The method of claim 7, wherein said step of contacting is followed by one or both of a step of rinsing and a step of drying, prior to said enzymatic hydrolytic saccharification.

14. The method of claim 7, in which the amount of aqueous ammonia is present at a ratio of between 4:1 to 10:1 relative to the dry weight of the ozone treated biomass.

15. The method of claim 7, further comprising the steps of
retaining, filtering and acid precipitating ammonia liquor generated in said step of contacting, and
recovering soluble and/or insoluble lignin generated in said step of contacting.

16. The method of claim 20, wherein sugars obtained from said enzymatic hydrolytic saccharification are used for lipid production by oleaginous microorganisms.

17. A method of pretreating biomass prior to hydrolytic saccharification, comprising
mixing said biomass with an H₂O₂ solution prepared in water to form 0% to 90% (w/w) moisture content of biomass and,
exposing said biomass/H₂O₂ mixture to ozone, wherein said step of exposing is carried out at a temperature of 15° C. to 30° C., and then
contacting ozone-treated biomass with aqueous ammonia at a temperature of or below about 50° C.

18. The method of claim 15, wherein said method includes a step of sizing said biomass prior to said step of mixing.

19. The method of claim 15, wherein said step of exposing is carried out for a time period of from about 5 to about 20 minutes.

20. The method of claim 15, wherein said step of contacting is carried out for a time period of from about 3 to about 9 hours.

21. The method of claim 15, wherein said step of exposing is followed by one or both of a step of rinsing and a step of drying, prior to said step of contacting.

22. The method of claim 15, wherein said step of contacting is followed by one or both of a step of rinsing and a step of drying, prior to said hydrolytic saccharification.

23. The method of claim 15, wherein the aqueous ammonia is present at a ratio of from 4:1 to 10:1 relative to a dry weight of the ozone-treated biomass.

24. The method of claim 15, further comprising the steps of
retaining, filtering and acid precipitating ammonia liquor generated in said step of contacting, and
recovering soluble and/or insoluble lignin generated in said step of contacting.

25. The method of claim 15, wherein sugars obtained from said enzymatic hydrolytic saccharification are used for lipid production by oleaginous microorganism.
